(19) United States
(12) Patent Application Publication
(10) Pub. No.: US 2024/0284945 A1
(43) Pub. Date: Aug. 29, 2024

(54) PIGMENT FOR MEAT SUBSTITUTE COMPOSITIONS

(71) Applicant: Cargill, Incorporated, Wayzata, MN (US)

(72) Inventors: Hans H. LIAO, Plymouth, MN (US); Catherine Bradshaw POOR, Minneapolis, MN (US)

(73) Assignee: Cargill, Incorporated, Wayzata, MN (US)

(21) Appl. No.: 18/572,037

(22) PCT Filed: Jun. 27, 2022
(86) PCT No.: PCT/US2022/073178

§ 371 Date: Dec. 19, 2023

Related U.S. Application Data
(60) Provisional application No. 63/217,572, filed on Jul. 1, 2021.

(51) Int. Cl. A23L 5/43; A23J 3/16; A23J 3/18; A23J 3/22; C07K 14/195; C09B 61/00

(52) U.S. Cl. CPC A23L 5/43; A23J 3/16; A23J 3/18; A23J 3/227; C07K 14/195; C09B 61/00

(57) ABSTRACT

Disclosed herein are pigment compositions for meat substitutes and meat substitutes including such pigment compositions. The pigment compositions include a thermolabile AlacDFX variant polypeptide, e.g. a thermolabile DFX polypeptide including a sequence at least 80% identical to SEQ ID NO:1 and including a mutation at a position selected from the group consisting of I76, H68, E106, K58, E108, K90, I15, I16, I89, Q88, F102, Y80, Y7, and combinations thereof relative to SEQ ID NO:1. The pigment compositions provide a pink and/or red color to a meat substitute composition that transitions to a brown color after cooking.

### CROSS-REFERENCE TO RELATED APPLICATIONS

This application claims priority to U.S. Provisional Patent Application No. 63/217,572, filed Jul. 1, 2021, which is incorporated herein by reference in its entirety.

### REFERENCE TO A SEQUENCE LISTING SUBMITTED VIA EFS-WEB

The content of the ASCII text file of the sequence listing named “PT-1117-WO-PCT_ST25.txt” which is 10.5 kb in size created on Jun. 20, 2022 and electronically submitted via Patent Center herewith the application is incorporated by reference in its entirety.

### BACKGROUND

Demand for plant-based meat substitutes is increasing for a variety of reasons. Many consumers prefer meat substitute options that perform most similarly to animal meat, including wanting the color of the meat substitute to be comparable to animal meat color before and after cooking. Accordingly, there is a need for a pigment that can provide color to a meat substitute that is the same or similar to that of natural animal meat. A pigment derived from natural sources that can transition in color when the meat substitute is cooked is particularly desirable.

### SUMMARY

The present disclosure provides compositions comprising A thermolabile desulfoferrodoxin (DFX) non-heme iron-binding protein polypeptide comprising a sequence at least 80% identical to SEQ ID NO:1 and a mutation at a position selected from the group consisting of isoleucine (I) 76, histidine (H) 68, glutamate (E) 106, lysine (K) 58, E108, K90, I15, I16, leucine (L) 81, I89, glutamine (Q) 88, phenylalanine (F) 102, tyrosine (Y) 80, Y7, and combinations thereof relative to SEQ ID NO:1. The polypeptide may comprise a sequence at least 85%, at least 90%, at least 95%, at least 98%, or at least 99% identical to SEQ ID NO:1. The polypeptide may comprise a mutation at a position selected from the group consisting of I76, H68, and combinations thereof relative to SEQ ID NO:1. The polypeptide may comprise at least one of: (i) a substitution of I76 relative to SEQ ID NO:1 with F; (ii) a substitution of H68 relative to SEQ ID NO:1 with arginine (R); (iii) a substitution of E106, K58, E108, and/or K90 relative to SEQ ID NO:1 with glycine (G), alanine (A), proline (P), valine (V), L, I, methionine (M), F, Y, tryptophan (W), serine (S), threonine (T), cysteine (C), asparagine (N), or Q; (iv) a substitution of I15, I16, L81, and/or I89 relative to SEQ ID NO:1 with G, A, V, S, or C; (v) a substitution of Q88 relative to SEQ ID NO:1 with G, A, V, L, I, M, S, T, C, K, R, D, or E; and (vi) a substitution of F102, Y80, and/or F7 relative to SEQ ID NO:1 with G, A, V, S, T, C, N, Q, K, R, H, D, or E. The polypeptide may comprise a sequence at least 80%, at least 85%, at least 90%, at least 95%, at least 98%, or at least 99% identical to the sequence of at least one of SEQ ID NOs:4-9. In some aspects, when the polypeptide is heated at 80° C. for 20 minutes, absorbance of light at a wavelength of 506 nm decreases relative to the absorbance prior to heating. The absorbance is decreased by at least 20%, at least 25%, at least 30%, at least 35%, at least 40%, at least 45%, at least 50%, at least 55%, at least 60%, at least 65%, at least 70% or at least 75% relative to the absorbance prior to heating.

For example, the disclosure provides pigment compositions comprising a thermolabile DFX as described herein in an amount effective for increasing the red color of a raw or uncooked meat substitute. In some aspects, when the pigment is heated at 80° C. for 20 minutes absorbance at 506 nm is decreased relative to the absorbance at 506 nm prior to heating.

The disclosure also provides a meat substitute comprising a thermolabile DFX polypeptide or pigment composition as described herein and a non-meat protein. The red color of the meat substitute may decrease after cooking. The non-meat protein may be a plant-based protein selected from the group consisting of pulse protein, pea protein, soy protein, corn protein, and wheat protein. The non-meat protein may be a fungi-based mycoprotein. The meat substitute may comprise 0.01% to 6%, 0.05% to 5%, 0.1% to 3%, or 0.5% to 2% by weight of a thermolabile DFX polypeptide as described herein. The meat substitute may comprise between 50% and 80%, between 55% and 75%, or between 58% and 70% by weight of water. The meat substitute may comprise between 1% and 25%, between 1.5% and 20%, between 2% and 15%, between 2.5% and 10%, between 3% and 8%, or between 4% and 7% by weight of a lipid composition. The lipid composition may comprise coconut oil, palm oil, sunflower oil, soy oil, canola oil, or combinations thereof. The meat substitute may comprise between 2% and 30%, between 5% and 25%, between 8% and 20%, or between 10% and 19% by weight of a textured plant-based protein. The textured plant-based protein may comprise textured pulse protein, textured pea protein, textured soy flour, textured soy concentrate, textured wheat protein, potato protein, or combinations thereof. The meat substitute may comprise between 0.5% and 8%, between 1% and 6%, between 20% and 40%, or between 25% and 35% by weight of a powdered plant-based protein. The powdered plant-based protein may comprise pulse protein isolate, pea protein isolate, soy flour, soy isolate, soy concentrate, vital wheat gluten, potato protein, corn protein isolate, or combinations thereof. The meat substitute may comprise methylcelluose in an amount up to 2% by weight or between 0.1% and 2% by weight. The meat substitute may be free of any tissue derived animal protein. The meat substitute may be free of any animal-based protein.

The disclosure further provides a cell comprising an exogenous polynucleotide encoding a thermolabile DFX polypeptide comprising a sequence at least 80% identical to SEQ ID NO:1 and comprising a mutation at a position selected from the group consisting of I76, H68, E106, K58, E108, K90, I15, I16, I89, Q88, F102, Y80, Y7, and combinations thereof relative to SEQ ID NO:1. The polypeptide may comprise a sequence at least 85%, at least 90%, at least 95%, at least 98%, or at least 99% identical to SEQ ID NO:1. The polypeptide may comprise a mutation at a position selected from the group consisting of I76, H68, and combinations thereof relative to SEQ ID NO:1. The polypeptide may comprise at least one of: (i) a substitution of I76 relative to SEQ ID NO:1 with F; (ii) a substitution of H68 relative to SEQ ID NO:1 with arginine (R); (iii) a substitution of E106, K58, E108, and/or K90 relative to SEQ ID NO:1 with glycine (G), alanine (A), proline (P), valine (V), L, I, methionine (M), F, Y, tryptophan (W), serine (S), threonine (T), cysteine (C), asparagine (N), or Q; (iv) a substitution of I15, I16, L81, and/or I89 relative to SEQ ID NO:1 with G, A, V, S, or C; (v) a substitution of Q88 relative to SEQ ID NO:1 with G, A, V, L, I, M, S, T, C, K, R, D, or E; and (vi) a substitution of F102, Y80, and/or F7 relative to SEQ ID NO:1 with G, A, V, S, T, C, N, Q, K, R, H, D, or E. The polypeptide may comprise a sequence at least 80%, at least 85%, at least 90%, at least 95%, at least 98%, or at least 99% identical to the sequence of at least one of SEQ ID NOs:4-9. The cell may be a plant cell, a fungi cell, or an animal cell, for example an insect cell or an in vitro cultured mammalian or avian cell. As provided herein are meat substitute composition comprising said cells.

The disclosure further provides a plasmid comprising a polynucleotide encoding a thermolabile DFX polypeptide comprising a sequence at least 60%, at least 70%, at least 80%, at least 85%, at least 90%, at least 95%, or at least 99% identical to SEQ ID NO:1 and comprising a mutation at a position selected from the group consisting of I76, H68, E106, K58, E108, K90, I15, I16, I89, Q88, F102, Y80, Y7, and combinations thereof relative to SEQ ID NO:1.

The disclosure also provides a method for increasing the red color of a meat substitute, comprising adding a thermolabile DFX polypeptide comprising a sequence at least 80% identical to SEQ ID NO:1 and comprising a mutation at a position selected from the group consisting of I76, H68, E106, K58, E108, K90, I15, I16, I89, Q88, F102, Y80, Y7, and combinations thereof relative to SEQ ID NO:1 to a meat substitute comprising a non-meat protein prior to cooking the meat substitute.

The disclosure also provides a method for decreasing red color in a cooked meat substitute, comprising cooking a meat substitute comprising a non-meat protein and a thermolabile DFX polypeptide comprising a sequence at least 80% identical to SEQ ID NO:1 and comprising a mutation at a position selected from the group consisting of I76, H68, E106, K58, E108, K90, I15, I16, I89, Q88, F102, Y80, Y7, and combinations thereof relative to SEQ ID NO:1, whereby the red color of the cooked meat substitute is reduced relative to red color of the meat substitute prior to cooking. In some aspects, when heated at 130° C. for 90 seconds the a* value of L*a*b* colorimetry of the meat substitute decreases by at least 5, 10, 15, 20, 25, 30, 35, 40, 45, or 50%.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed in the present document.

FIG. 1 shows dimer stabilization in AlacDFX by the salt bridge (dotted lines) formed between K90 of the first monomer (chain A) and E108 of the second monomer (chain B).

FIG. 2 shows dimer stabilization in AlacDFX by hydrophobic interactions (dotted lines) between I15 of the first monomer (chain A) and I16 of the second monomer (chain B).

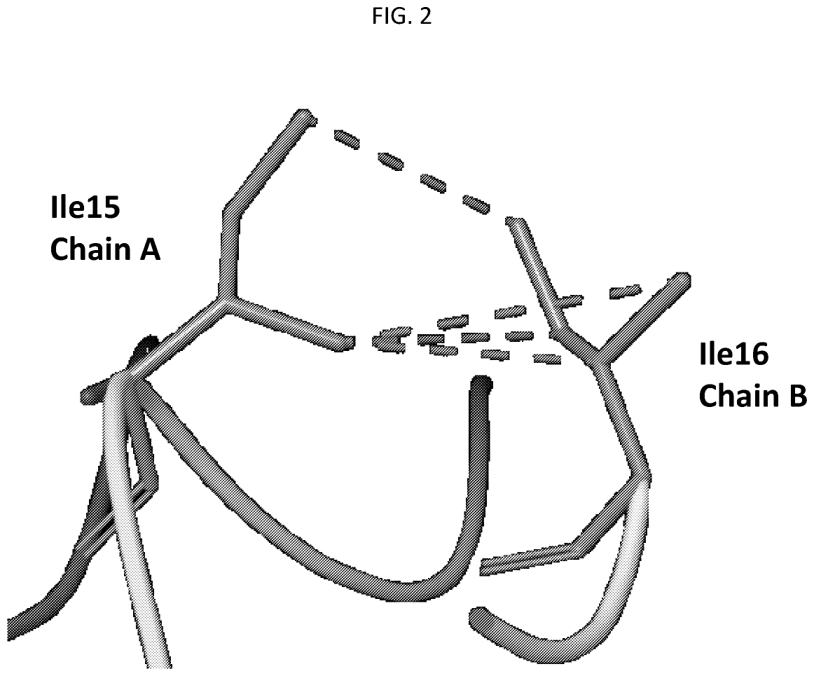

FIG. 3 shows dimer stabilization in AlacDFX by pi-pi stacking interactions (dotted lines) between Q88 of the first monomer (chain A) and Q88 of the second monomer (chain B).

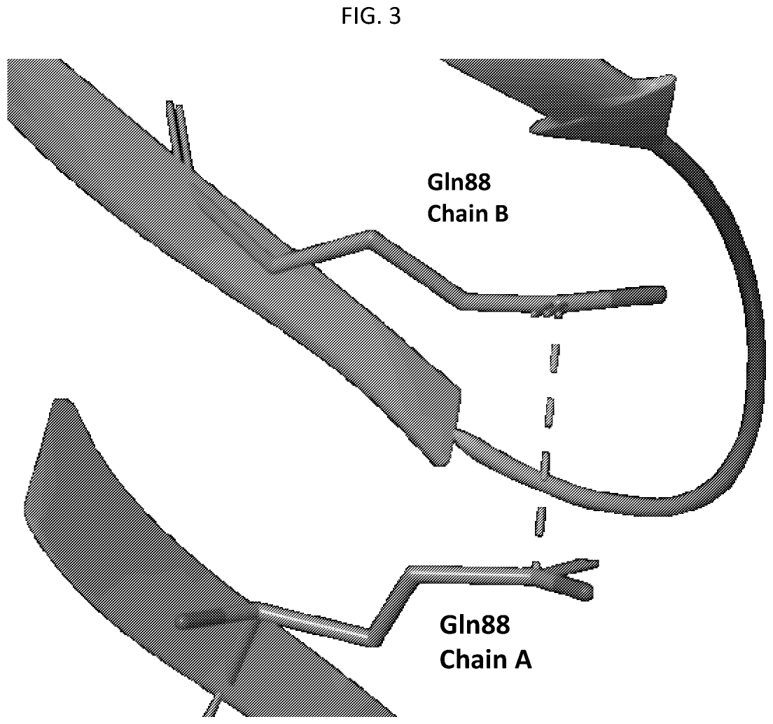

### DETAILED DESCRIPTION

Described herein are pigment compositions for meat substitutes that contain a thermolabile non-heme iron-binding protein. Thermolabile AlacDFX mutants may be used in a pigment composition having a similar pink/red color to raw animal meat before cooking, but the mutations in the AlacDFX protein makes the pigment composition susceptible to degradation during heating. This degradation of the pigment composition causes the pigment to have a substantially reduced color or become colorless after heating. Accordingly, meat substitutes containing an effective amount of this pigment composition will transition from a red color when raw to a brown or less red color when cooked. In an aspect, the brown color occurs because the pigment composition in the meat substitute becomes at least partially colorless during heating, which allows the brown color resulting from Maillard reactions involving other components of the meat substitute to become more visible than with other pigments used for meat substitutes.

Unless defined otherwise, all technical and scientific terms used herein have the same meaning as commonly understood by one skilled in the art to which this invention belongs. As used herein, each of the following terms has the meaning associated with it as defined below.

As used herein, the terms “meat substitute” and “meat substitute composition” are used interchangeably and refer to compositions that mimic the general appearance, nutritional content, and/or taste of natural animal meat or natural animal meat compositions without containing as the majority component tissues or cells from a whole, living vertebrate animal. For example, the meat substitute may be free of, or contain as a minor component, naturally-occurring animal muscle, adipose, or satellite cells from muscle tissues harvested from a whole vertebrate animal (e.g., a cow, a sheep, a pig, a chicken, a turkey, etc.). In some aspects, the meat substitute is free of any animal cells, e.g., any in vivo derived or in vitro cultured animal cells. In some aspects, the meat substitute is free of any animal-based protein including milk and egg proteins.

The meat substitutes and meat substitute compositions described herein include non-meat proteins, plant-based proteins (e.g., pulse protein, pea protein, soy protein, wheat protein, chickpea protein, corn protein, and the like), fungal-based proteins (e.g., mycoproteins derived from fungi such as Fusarium venenatum and the like), in vitro cultured animal cells (e.g., cultured muscle cells, satellite cells, adipose cells, and the like), insect proteins, or combinations thereof. The meat substitute can comprise plant-based proteins including, but not limited to, pea protein, soy protein, wheat protein, chickpea protein, and corn protein. The meat substitute can comprise fungal based proteins including, but not limited to, mycoproteins from Fusarium venenatum. The meat substitute may comprise fungal or microbial biomass comprising the fungal-based protein. The meat substitute can comprise in vitro cultured animal cells including, but not limited to, muscle cells, satellite cells, and adipose cells grown, differentiated and propagated using, for example, fermentation, a bioreactor, scaffold-seeded cell culture, or other artificial methods. The meat substitute can comprise a combination of two or more of plant-based protein, fungal-based proteins, insect proteins and in vitro cultured animal cells. For example, a meat substitute may include a pea protein and a fungal mycoprotein, a soy protein and a cultured bovine muscle cell, a cultured avian adipocyte and a fungal mycoprotein, or any other combination of plant-base protein, fungal-based protein, insect proteins, and in vitro cultured animal cells.

In some aspects, the meat substitute comprises plant-based proteins, fungal-based proteins, or combinations thereof and is free of any animal-based proteins or cells. In some aspects, the meat substitute comprises plant-based proteins, fungal-based proteins, insect proteins, and combinations thereof and is free of and any vertebrate animal-based cells or proteins. In some aspects, the meat substitute comprises plant-based proteins and is free of fungal-based, insect, or animal-based cells or proteins. In some aspects, the meat substitutes comprise fungal-based proteins and is free of plant-based, insect, and animal-based cells and proteins. In aspect, the meat substitute comprises insect proteins and is free of plant-based, fungal-based, and animal-based cells and proteins. In some aspects, the meat substitute comprises in vivo cultured animal cells and is free of plant-based proteins, fungal-based proteins, insect proteins, and in vivo whole animal derived tissues, cells, and proteins.

In some aspects, the meat substitute can mimic a beef product, e.g., ground beef, steak, beef jerky, beef ribs, beef patties, beef sausages, and the like. In some aspects, the meat substitute can mimic a pork product, e.g., ground pork, pork chops, ham, smoked pork, bacon, pork sausage, pork patties, pork ribs, and the like. In some aspects, the meat substitute can mimic a chicken product, e.g., ground chicken, chicken breast, check legs, chicken thighs, chicken wings, chicken patties, chicken tenders, chicken nuggets, chicken sausage, and the like. In some aspects, the meat substitute can mimic a turkey product, e.g., ground turkey, turkey sausage, turkey patties, and the like. In some aspects, the meat substitute can mimic a shellfish product, e.g., crab, lobster, shrimp, crayfish, clams, scallops, oysters, mussels, and the like. In some aspects, the meat substitute can mimic a cured, salted, or processed meat product, e.g., charcuterie, salami, summer sausage, prosciutto, bologna, kielbasa, and the like.

As used herein, the term “non-meat protein” refers to protein sourced from plants, fungus, insects, dairy products, or in vitro cultured animal cells, and excludes in vivo vertebrate animal derived tissues, cells, or proteins. For example, non-meat proteins may include plant-based proteins, fungal-based proteins, insect proteins, milk proteins (e.g., casein and whey), proteins from in vitro cultured animal cells, or combinations thereof.

As used herein, the terms “polypeptide” and “peptide” are used interchangeably and refer to the collective primary, secondary, tertiary, and quaternary amino acid sequence and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| Amino Acid | Three letter symbol | One letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, “AlacDFX” refers to the non-heme iron-binding desulfoferrodoxin from Anaerotignum lactatifermentans. GenBank ID AOA1M6LOQ2. The wild-type polypeptide sequence for AlacDFX is provided as SEQ ID NO:1.

SEQ ID NO: 1
MKAPRFFICKHCKNIITMVEDKGVPVVCCGEKMTELKANTSDGAG
EKHVPVVQVEGSKVTVKVGEVTHPMLEEHHIAWIYLETSQGGQIK
YLDHTGAPEAVFALAEGEQAVAAYEYCNLHGLWKAEI

As used herein, the term “thermolabile AlacDFX” refers to a AlacDFX polypeptide that, when heated at 80° C. for 20 minutes, has a decrease in absorbance at 506 nm relative to the absorbance prior to heating. In some aspects, after heating the thermolabile AlacDFX has an absorbance of less than 80%, less than 50%, or less than 20% of the absorbance at 506 nm prior to heating. Visually, the intensity of the red or pink color of the thermolabile AlacDFX may be reduced upon heating or the red or pink color may be completely absent following heating. Thermolabile AlacDFX polypeptides are variants of the thermostable wild-type AlacDFX polypeptide that include one or more mutations that destabilizes the AlacDFX polypeptide upon heating.

Thermolabile AlacDFX polypeptides suitable for use in the pigments and compositions described herein include thermolabile mutants of the AlacDFX protein of SEQ ID NO:1. The thermolabile AlacDFX mutants include one or more mutations that destabilize the polypeptide such that, when heated at 80° C. for 20 minutes, the absorbance at 506 nm and the red/pink color of the polypeptide is reduced relative to the color and absorbance prior to heating. The mutation may be a substitution, deletion, or insertion. Without wishing to be bound by any particular theory, aspect, or mode of action, mutations that destabilize the iron binding region or regions contributing to the structural integrity of the AlacDFX monomer or dimer will produce thermolabile AlacDFX polypeptides. See, for example, the analysis of the AlacDFX homology model presented in Example 2. The thermolabile AlacDFX polypeptide for use in the pigments and compositions (e.g., meat substitutes) described herein can be a polypeptide with a sequence at least 80%, at least 85%, at least 90%, at least 95%, or at least 98% identical to SEQ ID NO:1 and includes at least one mutation relative SEQ ID NO:1 that destabilizes the polypeptide such that, when heated at 80° C. for 20 minutes absorbance at 506 nm is reduced relative to the absorbance prior to heating.

Suitable destabilizing mutations in AlacDFX that from a thermolabile AlacDFX variant include, but are not limited to, a mutation at position I76, H68, E106, K58, E108, K90, I15, I16, L89, Q88, F102, Y80, Y7, or combinations thereof relative to SEQ ID NO:1. The destabilizing mutation may include a substitution at one or more positions selected from the group consisting of I76, H68, E106, K58, E108, K90, I15, I16, L89, Q88, F102, Y80, Y7, and combinations. The destabilizing mutation may include one or more substitutes selected from I76F and H68R.

The thermolabile AlacDFX polypeptide may include a I76F substitutions relative to SEQ ID NO:1 (SEQ ID NO:4). The thermolabile AlacDFX polypeptide may comprise a sequence at least 80%, at least 85%, at least 90%, at least 95%, or at least 98% identical to SEQ ID NO:4 and include the I76F mutation relative to SEQ ID NO:1.

SEQ ID NO: 4
MKAPRFFICKHCKNIITMVEDKGVPVVCCGEKMTELKANTSDGAG
EKHVPVVQVEGSKVTVKVGEVTHPMLEEHHFAWIYLETSQGGQIK
YLDHTGAPEAVFALAEGEQAVAAYEYCNLHGLWKAEI

The thermolabile AlacDFX polypeptide may include an H68R substitutions relative to SEQ ID NO:1 (SEQ ID NO:5). The thermolabile AlacDFX polypeptide may comprise a sequence at least 80%, at least 85%, at least 90%, at least 95%, or at least 98% identical to SEQ ID NO:5 and include the H68R mutation relative to SEQ ID NO:1.

SEQ ID NO: 5
MKAPRFFICKHCKNIITMVEDKGVPVVCCGEKMTELKANTSDGAG
EKHVPVVQVEGSKVTVKVGEVTRPMLEEHHIAWIYLETSQGGQIK
YLDHTGAPEAVFALAEGEQAVAAYEYCNLHGLWKAEI

The thermolabile AlacDFX polypeptide may include a substitution at one or more of positions E106, K58, E108, and K90 relative to SEQ ID NO:1 (SEQ ID NO:6). The thermolabile AlacDFX polypeptide may comprise a sequence at least 80%, at least 85%, at least 90%, at least 95%, or at least 98% identical to SEQ ID NO:6 and include a substitution at one or more of positions E106, K58, E108, and K90 relative to SEQ ID NO:1.

SEQ ID NO: 6
MKAPRFFICKHCKNIITMVEDKGVPVVCCGEKMTELKANTSDGAG
EKHVPVVQVEGSX₁VTVKVGEVTHPMLEEHHIAWIYLETSQGGQI
X₂YLDHTGAPEAVFALAX₃GX₄QAVAAYEYCNLHGLWKAEI

wherein X₁=K, G, A, P, V, L, I, M, F, Y, W, S, T, C, N, or Q, X₂=K, G, A, P, V, L, I, M, F, Y, W, S, T, C, N, or Q, X₃=E, G, A, P, V, L, I, M, F, Y, W, S, T, C, N, or Q, and X₄=E, G, A, P, V, L, I, M, F, Y, W, S, T, C, N, or Q, wherein if X₁ is K, X₂ is K, and X₄ is E, X₃ cannot be E, if X₁ is K, X₂ is K, and X₃ is E, X₄ cannot be E, if X₂ is K, X₃ is E, and X₄ is E, X₁ cannot be K, and if X₁ is K, X₃ is E, and X₄ is E, X₂ cannot be K.

The thermolabile AlacDFX polypeptide may include a substitution at one or more of positions I15, I16, I89 relative to SEQ ID NO:1 (SEQ ID NO:7). The thermolabile AlacDFX polypeptide may comprise a sequence at least 80%, at least 85%, at least 90%, at least 95%, or at least 98% identical to SEQ ID NO:7 and include a substitution at one or more of positions I15, I16, I89 relative to SEQ ID NO:1.

SEQ ID NO: 7
MKAPRFFICKHCKNXXTMVEDKGVPVVCCGEKMTELKANTSDGAG
EKHVPVVQVEGSKVTVKVGEVTHPMLEEHHIAWIYLETSQGGQXK
YLDHTGAPEAVFALAEGEQAVAAYEYCNLHGLWKAEI

wherein X=I, L, G, A, V, S, or C, and at least one X is not I or L.

The thermolabile AlacDFX polypeptide may include a substitution at positions Q88 relative to SEQ ID NO:1 (SEQ ID NO:8). The thermolabile AlacDFX polypeptide may comprise a sequence at least 80%, at least 85%, at least 90%, at least 95%, or at least 98% identical to SEQ ID NO:8 and include a substitution at position 88 relative to SEQ ID NO:1.

SEQ ID NO: 8
MKAPRFFICKHCKNIITMVEDKGVPVVCCGEKMTELKANTSDGAG
EKHVPVVQVEGSKVTVKVGEVTHPMLEEHHIAWIYLETSQGGXIK
YLDHTGAPEAVFALAEGEQAVAAYEYCNLHGLWKAEI

wherein X=G, P, C, S, A, M, T, K, L, V, or I

The thermolabile AlacDFX polypeptide may include a substitution at one or more of positions F102, Y80, and F7 relative to SEQ ID NO:1 (SEQ ID NO:9). The thermolabile AlacDFX polypeptide may comprise a sequence at least 80%, at least 85%, at least 90%, at least 95%, or at least 98% identical to SEQ ID NO:9 and include a substitution at one or more of positions F102, Y80, and F7 relative to SEQ ID NO:1.

SEQ ID NO: 9
MKAPRFXICKHCKNIITMVEDKGVPVVCCGEKMTELKANTSDGAG
EKHVPVVQVEGSKVTVKVGEVTHPMLEEHHIAWIXLETSQGGQIK
YLDHTGAPEAVXALAEGEQAVAAYEYCNLHGLWKAEI

wherein X=F, Y, G, A, V, S, T, C, N, Q, K, R, H, D, or E, and at least one X is not Y or F.

Variants or sequences having substantial identity or homology with the polypeptides described herein can be utilized in the practice of the disclosed pigments, compositions, and methods. Such sequences can be referred to as variants or modified sequences. That is, a polypeptide sequence can be modified yet still retain the ability to exhibit the desired activity. Generally, the variant or modified sequence may include or greater than about 45%, 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. There phrases also refer to DNA or RNA of natural or synthetic origin, which may be single-stranded or double-stranded and may represent the sense or the antisense strand. The DNA polynucleotides may be a cDNA or a genomic DNA sequence.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (i.e., polynucleotides encoding an AlacDFX polypeptide) may be codon-optimized for expression in a particular cell including, without limitation, a plant cell, bacterial cell, fungal cell, or animal cell. While polypeptides encoded by polynucleotide sequences found in Anaerotignum lactatifermentans are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

Also provided herein are polynucleotides encoding a thermolabile AlacDFX polypeptide. The polynucleotide may encode any of the thermolabile AlacDFX polypeptides described herein, for example, the polynucleotide may encode a polypeptide at least 80%, at least 85%, at least 90%, at least 95%, or at least 98% identical to SEQ ID NO:1 that includes a mutation at position I76, H68, E106, K58, E108, K90, I15, I16, L89, Q88, F102, Y80, Y7, or combinations thereof relative to SEQ ID NO:1.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding the thermolabile EforRed polypeptide. As used herein, the term “vector” refers to a polynucleotide capable of transporting another polynucleotide to which it has been linked. The vector may be a plasmid, which refers to a circular double-stranded DNA loop into which additional DNA segments may be integrated.

Cells including any of the polynucleotides, constructs, or vectors described herein are also provided. The cell may be a procaryotic cell or a eukaryotic cell. Suitable procaryotic cells include bacteria cell, for example, Escherichia coli and Bacillus subtilis cells. Suitable eukaryotic cells include, but are not limited to, fungal cells, plant cells, and animal cells. Suitable fungal cells include, but are not limited to, Fusarium venenatum, Pichia pastoris, Saccharomyces cerevisiae, Kluyveromyces lactis, Yarrowia lipolytica, Trichoderma reesei, Issatchenkia orientalis, and Aspergillus niger cells. Suitable plant cells include, but are not limited to, a pea cell (Pisum sativum), a corn cell (Zea mays), a soybean cell (Glycine max), and a wheat cell (Triticum sp.). Suitable animal cells include, but are not limited to, muscle cells (e.g., myocytes, myoblasts, myosatellite, and satellite cells) and fat cells (e.g., adipocytes or adipocyte progenitor cells such as mesenchymal stem cells). Suitable animal cells may be mammalian (e.g., bovine, porcine, and ovine), avian (e.g., poultry), crustacean (e.g., shrimp, lobster, and crab), mollusk (e.g., clam, mussel, scallop, and oyster) or insect cells. In some aspects, the cell is an edible mushroom cell, which refers to a mushroom that is safe for human consumption. For example, the edible mushroom cell can be a Fusarium venenatum, Agaricus bisporus, Lentinula edodes, or Volvariella volvacea cell.

Described herein are pigment compositions containing a thermolabile AlacDFX, and meat substitutes including such pigment compositions. The pigment compositions disclosed herein can be used to provide color to a meat substitute that is similar to the color of natural animal meat when raw. Further, these pigment compositions change color upon heating and can provide an overall color change to the entire meat substitute composition that mimics the effects of cooking on natural animal meat. In an aspect, the pigment composition provides a pink and/or red color to raw, uncooked meat substitute that transitions to a brown, white, colorless, or less red color after cooking the meat substitute.

The pigment composition itself loses its pink or red color as it is cooked due to degradation and may become colorless if enough degradation occurs. Accordingly, the brown color of a cooked meat substitute is not necessarily due to the pigment composition turning brown in color, but instead due to the pigment composition losing its reddish color. The degraded pigment composition in the cooked meat substitute no longer masks the other colors of the meat substitute and the brown colors associated with Maillard reactions in the meat substitute become more apparent.

The redness of the pigment composition is reduced substantially or eliminated when heated to a temperature within a range typically used for cooking meat. The pigment composition changes from a pink and/or red color to a less-pink/red color or becomes substantially colorless when heated at 80° C. for 20 minutes. The pigment composition can be used to change the color of a meat substitute from a pink and/or red color to a brown color and/or less pink/red color, as exhibited by heating a meat substitute including the pigment composition at 80° C. for 20 minutes.

The changes in color of a pigment composition sample can be measured using a Hunter Colorimeter and reported as a relative percent change in visible light absorbance after heating as compared to the sample prior to heating. When the thermolabile AlacDFX, the pigment composition, or the meat substitute is heated on a hot plate at 130° C. for 90 seconds, the a* value of L*a*b* colorimetry of the pigment composition decreases relative to the a* value prior to heating. The a* value may decrease by at least 5%, at least 10%, at least 15%, at least 20%, at least 25%, at least 30%, at least 35%, at least 40%, at least 45%, or at least 50%. Likewise, when the thermolabile AlacDFX, the pigment composition, or the meat substitute is heated at 80° C. for 20 minutes the absorbance of light at a wavelength of 506 nm is decreased relative to the absorbance prior to heating. The absorbance at 506 nm may decrease by at least 5%, at least 10%, at least 15%, at least 20%, at least 25%, at least 30%, at least 35%, at least 40%, at least 45%, or at least 50%.

The pigment compositions described herein include a thermolabile variant of the AlacDFX polypeptide. The thermolabile variant of AlacDFX polypeptide in the pigment composition may be any thermolabile valiant described herein. For example, the pigment composition may include a polypeptide with a sequence at least 80%, at least 85%, at least 90%, at least 95%, or at least 98% identical to SEQ ID NO:1 and includes at least one mutation relative SEQ ID NO:1 that destabilizes the polypeptide such that, when heated at 80° C. for 20 minutes absorbance at 506 nm is reduced relative to the absorbance prior to heating. The pigment compositions may include a polypeptide with a sequence at least 80%, at least 85%, at least 90%, at least 95%, or at least 98% identical to SEQ ID NO:1 with a mutation in at least one position selected from the group consisting of I76, H68, E106, K58, E108, K90, I15, I16, L89, Q88, F102, Y80, Y7, or combinations thereof relative to SEQ ID NO:1. The pigment compositions may include a polypeptide with a sequence at least 80%, at least 85%, at least 90%, at least 95%, or at least 98% identical to SEQ ID NO:1 that includes a substitution selected from I76F, H68R, and combinations thereof relative to SEQ ID NO:1.

The pigment compositions may include a polypeptide with a sequence at least 80%, at least 85%, at least 90%, at least 95%, or at least 98% identical to SEQ ID NO:4 and including the I76F substitution relative to SEQ ID NO:1. The pigment compositions may include a polypeptide with a sequence at least 80%, at least 85%, at least 90%, at least 95%, or at least 98% identical to SEQ ID NO:5 and including the H68R substitution relative to SEQ ID NO:1. The pigment compositions include a polypeptide with a sequence at least 80%, at least 85%, at least 90%, at least 95%, or at least 98% identical to SEQ ID NO:6 and including a mutation at a position selected from E106, K58, E108, K90, and combinations thereof relative to SEQ ID NO:1. The pigment compositions may include a polypeptide with a sequence at least 80%, at least 85%, at least 90%, at least 95%, or at least 98% identical to SEQ ID NO:7 and including a mutation at a position selected from I15, I16, I89, and combinations thereof relative to SEQ ID NO:1. The pigment compositions may include a polypeptide at least 80%, at least 85%, at least 90%, at least 95%, or at least 98% identical to SEQ ID NO:8 and including a mutation at position Q88 relative to SEQ ID NO:1. The pigment compositions may include a polypeptide with a sequence at least 80%, at least 85%, at least 90%, at least 95%, or at least 98% identical to SEQ ID NO:9 and including a mutation at a position selected from F102, Y80, F7, and combinations thereof relative to SEQ ID NO:1.

The pigment composition can be included in a meat substitute at a level that provides increased or improved pink and/or red color in the meat substitute, while also providing increased or improved brown color in the meat substitute after cooking. In an aspect, the pigment composition is used at a level such that the thermolabile AlacDFX is at least 0.01%, 0.05%, 0.1%, 0.2%, 0.3%, 0.4%, 0.5%, 1.0%, 1.25%, or 1.5% on a wet (total) weight basis in a meat substitute composition. The pigment composition may be used at a level such that the thermolabile AlacDFX is in the range of 0.01% to 6%, 0.05% to 5%, 0.1% to 3%, or 0.5% to 2% by weight in a meat substitute composition.

The pigment composition may additionally include a carrier or a diluent. The pigment composition may also include a blend of the AlacDFX polypeptide with another color or pigment. For example, the pigment composition may include the AlacDFX polypeptide and a fruit or vegetable extract-based pigment composition.

The pigment composition described herein can be used as a pigment in any meat substitute composition. In general, a meat substitute composition describe herein includes a non-meat protein (e.g., a plant-based protein), and optionally includes water, a lipid composition, fiber, starch, a gelling agent (e.g., methylcellulose), a preservative, a flavor, or combinations thereof. The meat substitute may be in a form that mimics a ground and formed meat (e.g., ground beef, sausage, or another meat product in which the raw meat has been ground and reformed), a deli or emulsified meat (e.g., hot dogs, bologna, and other processed meats), or a whole muscle (e.g., chicken breast, steak, and the like that are whole muscles from an animal). The meat substitute may include a textured plant-based protein, a powdered plant-based protein, a plant-based protein isolate, or combinations thereof. The meat substitute may include between 2% and 30%, between 5% and 25%, between 8% and 20%, or between 10% and 19% by weight of a textured plant-based protein. The meat substitute may include between 0.5% and 8%, between 1% and 6%, between 20% and 40%, or between 25% and 35% by weight of a powdered plant-based protein or plant-based protein isolate.

As used herein, “textured protein” and “textured plant-based protein” are used interchangeably and refer to edible food ingredients processed from an edible protein sources and characterized by having a structural integrity and identifiable structure such that individual units, appearing as fibers, shreds, chunks, bits, granules, slices, and the like, will withstand hydration and cooking or other procedures used in the production of food for consumption. In general, textured plant-based proteins are used to mimic the texture of meat and bind water in the meat substitute compositions. Edible protein sources from which textured proteins are produced may include, but are not limited to, legumes (e.g., pulse protein), pea, soy, corn, wheat, chickpea, potato, and the like. Textured proteins may include, but are not limited to, textured pulse protein, textured pea protein, textured soy flour, textured soy concentrate, textured wheat protein, textured potato protein, or combinations thereof. Methods for protein texturization and known and described in the art, and may include, for example, high temperature and pressure extrusion, spinning, freeze texturization, chemical or enzymatic texturization, and the like.

Meat substitutes described herein may also include a non-textured plant-based protein, for example, a powdered plant-based protein, a plant-based protein isolate, a plant-protein based flour, a plant-protein concentrate, combinations thereof, and the like. Powdered plant-based proteins and plant-based protein isolates can include soluble forms of plant-based proteins used as food ingredients. Non-textured plant-based proteins may include, but are not limited to, pea protein, defatted soy flour, defatted soy isolate, soy concentrate, vital wheat gluten, potato protein, corn protein isolate, or combinations thereof.

The meat substitute may include a high moisture textured plant-based protein. In general, high moisture textured plant-based proteins are hydrated prior to addition to a meat substitute formulation and therefore may constitute a higher percentage thereof on a weight basis of the meat substitute composition. For example, the meat substitute composition may include between 25% and 98%, between 50% and 95%, or between 60% and 90% by weight of a high moisture textured plant-based protein.

The meat substitute may include one or more lipid compositions, for example a fat, an oil, or combinations thereof. In general, fats refer to lipid compositions that are solid at room temperature, whereas oils are liquid at room temperature. The lipid compositions may include saturated fatty acids (also referred to as “saturated fats”), unsaturated fatty acids (also referred to as “unsaturated fats”), or combinations thereof. The lipid composition may include, but are not limited to, vegetable oil, coconut oil, palm oil, sunflower oil, soy oil, canola oil, or combinations thereof. The meat substitute composition may include between 1% and 25%, between 1.5% and 20%, between 2% and 15%, between 2.5% and 10%, between 3% and 8%, or between 4% and 7% by weight of a lipid composition.

In some aspects, the meat substitute may include a lipid mimetic instead of or in addition to a lipid composition described herein. As used herein, the term “lipid mimetic” refers to a compound or composition that mimics the form, function, texture, mouthfeel, and taste of a lipid composition when used as a food ingredient. A lipid mimetic for use in the meat substitute composition describe herein may include, but is not limited to, a fiber, a starch, a carbohydrate, a protein, or combinations thereof. In some aspect, the lipid mimetic may be a plant extract. The meat substitute composition may include between 1% and 25%, between 1.5% and 20%, between 2% and 15%, between 2.5% and 10%, between 3% and 8%, or between 4% and 7% by weight of a lipid mimetic. When the lipid mimetic is used in combination with a lipid composition, the meat substitute may include between 1% and 25%, between 1.5% and 20%, between 2% and 15%, between 2.5% and 10%, between 3% and 8%, or between 4% and 7% by weight of the combination of the lipid mimetic and the lipid composition.

The meat substitute may include water. For example, the meat substitute may include between 50% (wt) and 80% (wt), between 55% (wt) and 75% (wt), or between 58% (wt) and 70% (wt) of water.

The meat substitute may include fiber. The fiber may include, but is not limited to, pectin, apple fiber, psyllium, flax fiber, rice bran extract, Konjac flour, and the like. The meat substitute may include between 0.1% (wt) and 3% (wt), between 0.1% (wt) and 2% (wt), or between 0.5% (wt) and 2% (wt) of fiber. The meat substitute may include fiber in an amount up to 1% (wt), up to 1.5% (wt), up to 2% (wt), up to 2.5% (wt), or up to 3% (wt).

The meat substitute may include starch. The starch may include a pregelatinized starch, a modified starch, or combinations thereof. The starch may include, but is not limited to, corn starch, potato starch, tapioca starch, and the like. The meat substitute may include between 0.1% (wt) and 3% (wt), between 0.1% (wt) and 2% (wt), or between 0.5% (wt) and 2% (wt) of starch. The meat substitute may include starch in an amount up to 1% (wt), up to 1.5% (wt), up to 2% (wt), up to 2.5% (wt), or up to 3% (wt).

The meat substitute may include a gelling agent. The gelling agent may include, but is not limited to, methylcellulose, egg white protein, casein, pectin, hydrocolloids (e.g. guar gum, xanthan gum, locust bean gum, and the like), soy protein, canola protein, a crosslinking enzyme (e.g., transglutaminase), and combinations thereof. The meat substitute may include between 0.1% (wt) and 3% (wt), between 0.1% (wt) and 2% (wt), or between 0.5% (wt) and 2% (wt) of a gelling agent. The meat substitute may include a gelling agent in an amount up to 1% (wt), up to 1.5% (wt), up to 2% (wt), up to 2.5% (wt), or up to 3% (wt).

In some aspects, the gelling agent is methylcellulose. The meat substitute may include between 0.1% (wt) and 3% (wt), between 0.1% (wt) and 2% (wt), or between 0.5% (wt) and 2% (wt) of methylcellulose. The meat substitute may include methylcellulose in an amount up to 1% (wt), up to 1.5% (wt), up to 2% (wt), up to 2.5% (wt), or up to 3% (wt).

The meat substitute may include a preservative. For example, the meat substitute may include a preservative such as potassium sorbate, cultured dextrose, vinegar, and the like.

The meats substitute may include a pigment. Pigments for meat substitute compositions are known and described in the art and may include, but are not limited to, fruit and vegetable extracts (e.g., beet juice and beet extracts), heme containing proteins, and the like.

The meat substitute may include a flavor or seasoning. For example, the meat substitute may include a natural or artificial flavor and/or a seasoning. Seasonings may include, but are not limited to, yeast extract, spices, paprika, garlic (e.g., garlic powder, minced garlic, dehydrated garlic), onion (e.g., onion powder, minced onion, dehydrated onion), oregano, parsley, sweetener, salt (e.g., sodium chloride or potassium chloride), cayenne, chili powder, cumin, ginger, and the like.

The meat substitute may include a sweetener. Suitable sweeteners are known and described in the art. The sweetener can be at least one of a non-caloric sweetener or a caloric sweetener. The sweetener can be any type of sweetener, for example, a sweetener obtained from a plant or plant product, or a physically or chemically modified sweetener obtained from a plant, or a synthetic sweetener.

An exemplary, but non-limiting, meat substitute composition is a composition which comprises: plant protein (e.g., textured pea protein and/or pea protein), water, vegetable oil, flavor ingredients, salt, sugar, binders, and the pigment composition described herein. The pigment composition described herein can also be used in food applications other than meat substitutes.

Meat substitutes described herein may include one or more cells comprising an exogenous polynucleotide encoding a thermolabile AlacDFX polypeptide as described herein. For example, the meat substitutes may include a fungal, plant, or animal cell as described herein comprising an exogenous polynucleotide encoding a thermolabile AlacDFX polypeptide described herein.

Also provided herein is a method for increasing the red color of a meat substitute. The method for increasing the red color of a meat substitute includes adding a thermolabile AlacDFX polypeptide to a meat substitute prior to cooking the meat substitute, wherein the red color of the uncooked meat substitute is increase relative to the meat substitute without the thermolabile AlacDFX polypeptide. The method may also include adding a thermolabile AlacDFX polypeptide to a non-meat protein to form a meat substitute with increased red color relative to the non-meat protein without the AlacDFX polypeptide. The thermolabile AlacDFX polypeptide may be any thermolabile AlacDFX polypeptide as described herein. For example, the thermolabile AlacDFX polypeptide to be added to the meat substitute may comprise a sequence at least 80% identical to SEQ ID NO:1 and comprise a mutation at a position selected from the group consisting of I76, H68, E106, K58, E108, K90, I15, I16, L89, Q88, F102, Y80, Y7, and combinations thereof, relative to SEQ ID NO:1.

Also provided is a method for decreasing red color in a cooked meat substitute. The method for decreasing the red color in a cooked meat substitute includes cooking a meat substitute comprising a non-meat protein and a thermolabile AlacDFX polypeptide, whereby red color of the cooked meat substitute is reduced relative to the meat substitute prior to cooking. The thermolabile AlacDFX polypeptide may be any thermolabile AlacDFX polypeptide as described herein. For example, the thermolabile AlacDFX polypeptide to be added to the meat substitute may comprise a sequence at least 80% identical to SEQ ID NO:1 and comprise a mutation at a position selected from the group consisting of I76, H68, E106, K58, E108, K90, I15, I16, L89, Q88, F102, Y80, Y7, and combinations thereof, relative to SEQ ID NO:1.

### Examples

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1—Heat Stability of Desulfoferrodoxin

The desulfoferrodoxin from Anaerotignum lactatifermentans (AlacDFX; SEQ ID NO:1) has a red color that, when used in a meat substitute composition, imparts a red color similar to the color of uncooked or raw meat. However, AlacDFX is heat stable and does not lose its red color upon heating.

A library of AlacDFX mutants was generated using error-prone PCR. The error-prone PCR on the wild-type AlacDFX cDNA sequence (SEQ ID NO:2), which also included a region coding for a His6 tag and protease cleavage site (MGSSHIIHHSSGLVPRGSH, SEQ ID NO:3), was carried out with a nonproofreading DNA polymerase in the presence of Mn²⁺, the resulting polynucleotides were transformed into E. coli, and resulting red E. coli colonies were selected for further screening. The selected colonies were grown up on in 96-well liquid cultures, cells were lysed, and the lysate supernatant was removed from the cell debris. The absorbance of the lysate supernatant at 506 nm was measured before and after heating at 80° C. for 20 min. Lysates that demonstrated a decrease in A506 following heating were repeated in 50 ml cultures. Results for the lysates from the 50 ml cultures, along with characterization of the destabilizing mutation relative to SEQ ID NO:1, are shown in Table A. “% absorbance at 506 nm after heating” in Table A is the % absorbance at 506 nm after heating compared to the lysate prior to heating.

TABLE A
50 mL lysate thermal stability

| Lysate | % absorbance at 506 nm after heating | Mutation |
|---|---|---|
| 3D6 | 45% | I76F |
| 2E8 | 47% | H68R |

### Example 2—AlacDFX Homology Model and Stability Predictions

A homology model for the AlacDFX protein was built using the x-ray crystal structure of superoxide reductase (PDB ID 1Y07). This model was used to identify mutations that are expected to decrease thermal stability of AlacDFX.

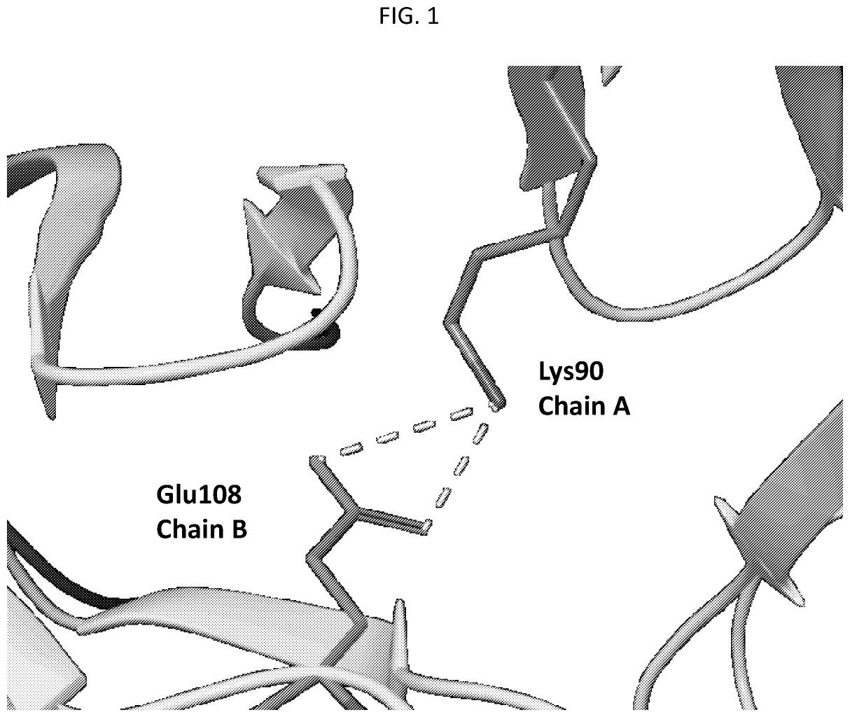

First, mutation of one or more of residues E106, K58, E108, and K90 will destabilize salt bridges between individual monomers within the dimer (FIG. 1). In the AlacDFX model, residues E106 and E108 from one monomer form salt bridges to residues K58 and K90, respectively, of the other monomer. Mutation of any one or more of these residues is expected to disrupt the quaternary structure of the protein resulting in a thermolabile AlacDFX protein with a decrease in absorbance at 506 nm upon heating. The mutations in one or more of E106, K58, E108, and K90 may be a non-conservative amino acid substitute, for example, a substitution that removes the charged side chain (e.g., substitution for G, A, P, V, L, I, M, F, Y, W, S, T, C, N, or Q), or a substitute that increases the bulk of the side chain (e.g., substitution for F, Y, or W).

Mutation of I15, I16, or L89, by substitution for a smaller (i.e., less bulky) residue will disrupt the dimerization interface. In the AlacDFX model, residues I15, I16, and L89 are located at the dimerization interface and the size of the side chains of these residues contribute to the stabilization of the dimer interface. Mutation of any one or more of these residues is expected to disrupt the quaternary structure of the protein resulting in a thermolabile AlacDFX protein with a decrease in absorbance at 506 nm upon heating. The mutations in one or more of I15, I16, or L89 may be a substitution for an amino acid with a smaller side chain, for example, G, A, S, or C.

Mutation of Q88 by substitution for a non-aromatic residue will disrupt pi-pi stacking interactions in the protein resulting in destabilization. In the model, Q88 has pi-pi stacking interactions with the Q88 residue from the other monomer of the dimer. Upon substitution of Q88 with G, P, C, S, A, M, T, K, L, V, or I, the sidechain-sidechain pi-pi stacking interactions will be disrupted resulting in a thermolabile AlacDFX protein with a decrease in absorbance at 506 nm upon heating.

The model of AlacDFX is characterized by a hydrophobic core formed by at least residues F102, Y80, and F7. Loosening this hydrophobic core, for example upon substitution for a smaller or charged amino acid (e.g., G, A, V, S, T, C, N, Q, K, R, H, D, or E) will result in a thermolabile AlacDFX protein with a decrease in absorbance at 506 nm upon heating.

1. A thermolabile desulfoferrodoxin (DFX) non-heme iron-binding protein polypeptide comprising a sequence at least 80% identical to SEQ ID NO:1 and a mutation at a position selected from the group consisting of isoleucine (I) 76, histidine (H) 68, glutamate (E) 106, lysine (K) 58, E108, K90, I15, I16, leucine (L) 81, I89, glutamine (Q) 88, phenylalanine (F) 102, tyrosine (Y) 80, Y7, and combinations thereof relative to SEQ ID NO:1.

2. (canceled)

3. The thermolabile DFX of claim 1, wherein the polypeptide comprises a mutation at a position selected from the group consisting of I76, H68, and combinations thereof relative to SEQ ID NO:1.

4. The thermolabile DFX of claim 1, wherein the polypeptide comprises at least one of:
(i) a substitution of I76 relative to SEQ ID NO:1 with F;
(ii) a substitution of H68 relative to SEQ ID NO:1 with arginine (R);
(iii) a substitution of E106, K58, E108, and/or K90 relative to SEQ ID NO:1 with glycine (G), alanine (A), proline (P), valine (V), L, I, methionine (M), F, Y, tryptophan (W), serine (S), threonine (T), cysteine (C), asparagine (N), or Q;
(iv) a substitution of I15, I16, L81, and/or I89 relative to SEQ ID NO:1 with G, A, V, S, or C;
(v) a substitution of Q88 relative to SEQ ID NO:1 with G, A, V, L, I, M, S, T, C, K, R, D, or E; and
(vi) a substitution of F102, Y80, and/or F7 relative to SEQ ID NO:1 with G, A, V, S, T, C, N, Q, K, R, H, D, or E.

5. The thermolabile DFX of claim 1, wherein the polypeptide comprises a sequence at least 80% identical to the sequence of at least one of SEQ ID NOs:4-9.

6. (canceled)

7. (canceled)

8. A pigment composition for a meat substitute comprising the thermolabile DFX polypeptide of claim 1 in an amount effective for increasing the red color of a raw or uncooked meat substitute.

9. (canceled)

10. A meat substitute comprising:
the thermolabile DFX polypeptide of claim 1, and
a non-meat protein.

11. (canceled)

12. The meat substitute of claim 10, wherein the non-meat protein is a plant-based protein selected from the group consisting of pulse protein, pea protein, soy protein, corn protein, and wheat protein.

13. (canceled)

14. The meat substitute of claim 10, wherein the meat substitute comprises 0.01% to 6% by weight of the thermolabile DFX polypeptide.

15. The meat substitute of claim 10, wherein the meat substitute comprises between 50% and 80% by weight of water.

16. The meat substitute of claim 10, wherein the meat substitute comprises between 1% and 25% by weight of a lipid composition comprising coconut oil, palm oil, sunflower oil, soy oil, canola oil, or combinations thereof.

17. (canceled)

18. The meat substitute of claim 10, wherein the meat substitute comprises between 2% and 30% by weight of a textured plant-based protein comprising textured pulse protein, textured pea protein, textured soy flour, textured soy concentrate, textured wheat protein, potato protein, or combinations thereof.

19. (canceled)

20. The meat substitute of claim 10, wherein the meat substitute comprises between 0.5% and 8% by weight of a powdered plant-based protein comprising pulse protein isolate, pea protein isolate, soy flour, soy isolate, soy concentrate, vital wheat gluten, potato protein, corn protein isolate, or combinations thereof.

21. (canceled)

22. The meat substitute of claim 10, wherein the meat substitute comprises methylcelluose in an amount up to 2% by weight or between 0.1% and 2% by weight.

23. The meat substitute of claim 10, wherein the meat substitute is free of any animal protein.

24. (canceled)

25. A method for increasing the red color of a meat substitute, comprising:
adding the thermolabile DFX polypeptide of claim 1 to a meat substitute comprising a non-meat protein prior to cooking the meat substitute.

26. A method for decreasing red color in a cooked meat substitute, comprising:
cooking a meat substitute comprising a non-meat protein and a thermolabile DFX polypeptide comprising a sequence at least 80% identical to SEQ ID NO:1 and comprising a mutation at a position selected from the group consisting of I76, H68, E106, K58, E108, K90, I15, I16, I89, Q88, F102, Y80, Y7, and combinations thereof relative to SEQ ID NO:1, whereby the red color of the cooked meat substitute is reduced relative to red color of the meat substitute prior to cooking.

27. The method of claim 26, wherein when heated at 130° C. for 90 seconds the a* value of L*a*b* colorimetry of the meat substitute decreases by at least 5%.

28. A cell comprising an exogenous polynucleotide encoding a thermolabile DFX polypeptide comprising a sequence at least 80% identical to SEQ ID NO:1 and comprising a mutation at a position selected from the group consisting of I76, H68, E106, K58, E108, K90, I15, I16, I89, Q88, F102, Y80, Y7, and combinations thereof relative to SEQ ID NO:1.

29. (canceled)

30. The cell of claim 28, wherein the polypeptide comprises a mutation at a position selected from the group consisting of I76, H68, and combinations thereof relative to SEQ ID NO:1.

31. The cell of claim 30, wherein the polypeptide comprises an I76F or H68R substitution relative to SEQ ID NO:1.

32. The cell of claim 28, wherein the polypeptide comprises at least one of:
(i) a substitution of I76 relative to SEQ ID NO:1 with F;
(ii) a substitution of H68 relative to SEQ ID NO:1 with arginine (R);
(iii) a substitution of E106, K58, E108, and/or K90 relative to SEQ ID NO:1 with glycine (G), alanine (A), proline (P), valine (V), L, I, methionine (M), F, Y, tryptophan (W), serine (S), threonine (T), cysteine (C), asparagine (N), or Q;
(iv) a substitution of I15, I16, and/or I89 relative to SEQ ID NO:1 with G, A, V, S, or C;
(v) a substitution of Q88 relative to SEQ ID NO:1 with G, A, V, L, I, M, S, T, C, K, R, D, or E; and
(vi) a substitution of F102, Y80, and/or F7 relative to SEQ ID NO:1 with G, A, V, S, T, C, N, Q, K, R, H, D, or E.

33. The cell of claim 28, wherein the thermolabile DFX polypeptide comprises a sequence at least 90% identical to the sequence of at least one of SEQ ID NOs:4-9.

34. The cell of claim 28, wherein the cell a plant cell, a fungal cell, an insect cell, or an in vitro cultured mammalian or avian cell.

35.-38. (canceled)

39. A meat substitute comprising the cell of claim 28.

40. (canceled)


SEQUENCE LISTING (9 sequences)

SEQ ID NO: 1 - protein, 127 aa, Anaerotignum lactatifermentans
MKAPRFFICKHCKNIITMVEDKGVPVVCCGEKMTELKANTSDGAGEKHVPVVQVEGSKVTVKVGEVTHPMLEEHHIAWIYLETSQGGQIKYLDHTGAPEAVFALAEGEQAVAAYEYCNLHGLWKAEI

SEQ ID NO: 2 - DNA, 444 bp, Anaerotignum lactatifermentans
atgggcagcagccatcatcatcatcatcacagcagcggcctggtgccgcgcggcagccatatgaaggcgccgcgcttctttatctgcaaacactgtaagaacatcatcaccatggtagaagataaaggcgtgccggtcgtctgttgcggtgaaaaaatgacagaattaaaagcgaatacctccgacggtgccggtgaaaaacacgtcccggttgtccaggtggaaggctctaaagtgactgtcaaagtgggtgaagtgactcaccctatgctggaagaacaccatatcgcttggatctacctggagacttcacaaggcggtcagattaaatacttagaccatacgggtgcgccggaggcagtctttgcactggccgagggcgaacaagcagtcgctgcatacgaatattgcaacttacatggattatggaaagccgaaatctaa

SEQ ID NO: 3 - protein, 20 aa, Artificial Sequence
  synthetic
MGSSHHHHHHSSGLVPRGSH

SEQ ID NO: 4 - protein, 127 aa, Artificial Sequence
  synthetic
MKAPRFFICKHCKNIITMVEDKGVPVVCCGEKMTELKANTSDGAGEKHVPVVQVEGSKVTVKVGEVTHPMLEEHHFAWIYLETSQGGQIKYLDHTGAPEAVFALAEGEQAVAAYEYCNLHGLWKAEI

SEQ ID NO: 5 - protein, 127 aa, Artificial Sequence
  synthetic
MKAPRFFICKHCKNIITMVEDKGVPVVCCGEKMTELKANTSDGAGEKHVPVVQVEGSKVTVKVGEVTRPMLEEHHIAWIYLETSQGGQIKYLDHTGAPEAVFALAEGEQAVAAYEYCNLHGLWKAEI

SEQ ID NO: 6 - protein, 127 aa, Artificial Sequence
  synthetic
  MISC_FEATURE (58)..(58): X is K, G, A, P, V, L, I, M, F, Y, W, S, T, C, N, or Q
  MISC_FEATURE (90)..(90): X is K, G, A, P, V, L, I, M, F, Y, W, S, T, C, N, or Q
  MISC_FEATURE (106)..(106): X is E, G, A, P, V, L, I, M, F, Y, W, S, T, C, N, or Q
  MISC_FEATURE (108)..(108): X is E, G, A, P, V, L, I, M, F, Y, W, S, T, C, N, or Q
MKAPRFFICKHCKNIITMVEDKGVPVVCCGEKMTELKANTSDGAGEKHVPVVQVEGSXVTVKVGEVTHPMLEEHHIAWIYLETSQGGQIXYLDHTGAPEAVFALAXGXQAVAAYEYCNLHGLWKAEI

SEQ ID NO: 7 - protein, 127 aa, Artificial Sequence
  synthetic
  MISC_FEATURE (15)..(16): X is I, L, G, A, V, S, or C
  MISC_FEATURE (15)..(89): X is I, L, G, A, V, S, or C; and at least one X is not I or L
  MISC_FEATURE (89)..(89): X is I, L, G, A, V, S, or C
MKAPRFFICKHCKNXXTMVEDKGVPVVCCGEKMTELKANTSDGAGEKHVPVVQVEGSKVTVKVGEVTHPMLEEHHIAWIYLETSQGGQXKYLDHTGAPEAVFALAEGEQAVAAYEYCNLHGLWKAEI

SEQ ID NO: 8 - protein, 127 aa, Artificial Sequence
  synthetic
  MISC_FEATURE (88)..(88): X is G, P, C, S, A, M, T, K, L, V, or I
MKAPRFFICKHCKNIITMVEDKGVPVVCCGEKMTELKANTSDGAGEKHVPVVQVEGSKVTVKVGEVTHPMLEEHHIAWIYLETSQGGXIKYLDHTGAPEAVFALAEGEQAVAAYEYCNLHGLWKAEI

SEQ ID NO: 9 - protein, 127 aa, Artificial Sequence
  synthetic
  MISC_FEATURE (7)..(7): X is F, Y, G, A, V, S, T, C, N, Q, K, R, H, D, or E
  MISC_FEATURE (7)..(102): X is F, Y, G, A, V, S, T, C, N, Q, K, R, H, D, or E; and at least one X is not Y or E
  MISC_FEATURE (80)..(80): X is F, Y, G, A, V, S, T, C, N, Q, K, R, H, D, or E
  MISC_FEATURE (102)..(102): X is F, Y, G, A, V, S, T, C, N, Q, K, R, H, D, or E
MKAPRFXICKHCKNIITMVEDKGVPVVCCGEKMTELKANTSDGAGEKHVPVVQVEGSKVTVKVGEVTHPMLEEHHIAWIXLETSQGGQIKYLDHTGAPEAVXALAEGEQAVAAYEYCNLHGLWKAEI